(19) United States
(12) Patent Application Publication
(10) Pub. No.: US 2008/0132878 A1
(43) Pub. Date: Jun. 5, 2008

(54) DEVICE FOR CELL DELIVERY INTO THE BRAIN OR BODY

(76) Inventor: Douglas KONDZIOLKA, Pittsburgh, PA (US)

Correspondence Address: REED SMITH LLP, P.O. BOX 488, PITTSBURGH, PA, 15230-0488, US

(21) Appl. No.: 11/940,868

(22) Filed: Nov. 15, 2007

Related U.S. Application Data
(60) Provisional application No. 60/861,836, filed on Nov. 30, 2006.

(51) Int. Cl. A61M 31/00; A61M 5/315

(52) U.S. Cl. 604/522; 604/218

(57) ABSTRACT

Apparatuses and methods for the effective implantation of viable cells into the body of a patient. Apparatuses and methods are disclosed that are particularly useful for the effective implantation of viable cells into a patient. The apparatuses preferably include a syringe with a small or zero dead volume, a cannula adapted for cell implantation, and a rotational chamber. The syringe is able to hold a cellular suspension for administration to a patient. The apparatuses and methods also preferably employ a small motor which is adapted to rotate the syringe, cannula, and rotational chamber slowly. That rotation reduces cell clumping in the cellular suspension and better maintains the cellular suspension during the implantation procedure. The apparatuses and methods are particularly well suited for the implantation of neurons into the brain of a patient. The apparatuses and methods may also be used to control the rate and volume of cell delivery during implantation precisely and in an automated manner. The present invention may also be used to delivery small volume fluid suspensions into tissues or cavities of the body. Through the cell or fluid delivery, the apparatuses of the present invention may be used to restore function of the target tissues in patients who have suffered ischemic, toxic, or traumatic injury to the target tissue.

### BACKGROUND OF THE INVENTION

1. Field of the Invention

The field of the present invention generally relates to methods and apparatuses for the sterile and effective implantation of cells to a target tissue.

2. Description of the Background

Stroke impacts millions of people worldwide with greater than 500,000 new patients per year in the United States. Although less than a third of strokes are fatal, approximately 60% of patients show significant residual impairments, and the prevalence of stroke-related morbidity will increase as the population ages because there are no therapies that reverse these effects. However, implantation of neural cells in the stroke penumbra may have therapeutic benefits by re-establishing neuronal circuits or enhancing functions of residual penumbral neurons.

Preliminary reports have demonstrated that transplantation of neurons derived from a clonal human teratocarcinoma cell line (hNT neurons; LAYTON BIOSCIENCE, Inc.) into the stroke penumbra in basal ganglia stroke patients results in measurable behavioral and cognitive improvements in some stroke patients. During those procedures, viable neurons are suspended in solution and placed directly into the stroke penumbra using a long cannula. Diversity in the results obtained in those studies may reflect inter-patient variability or differences in surgery between patients. Because the procedure is performed with the patient prone, the implantation cannula is held horizontally over the entire course of the implantation. During that time, the cell suspension tends to settle to one side of the cannula due to gravity, thereby creating an inconsistent formulation that is administered to patients. Attempts are made during surgery to rotate the syringe and cannula by hand, though concerns remain about the consistency of that procedure. No surgical tools are currently available that can accomplish the goal of administering a consistent formulation of cells to a patient over an extended period of time.

In addition, administration of small volumes of pharmaceutical formulations directly to target tissues has the potential to greatly increase their medical efficacy. Reproducible administration of precise volumes to the target tissue is likely to be important in obtaining optimal results. Currently, no surgical tools exist that provide the medical practitioner with that ability.

Thus, there has been a long-standing need in the medical community for devices for the consistent implantation of cells and delivery of fluids into a target tissue. Preferably, the device would be adapted to provide precise volumes and to maintain a uniform cellular suspension during the implantation procedure. The present invention satisfies these needs.

### BRIEF DESCRIPTION OF THE DRAWINGS

For the present invention to be clearly understood and readily practiced, the present invention will be described in conjunction with the following figures, wherein like reference characters designate the same or similar elements, which figures are incorporated into and constitute a part of the specification, wherein:

FIG. 1 depicts a cannula that may be used in the context of the present invention;

FIG. 2 displays the manner in which the cannula of FIG. 1 may be connected to a syringe; and

FIG. 3 shows a presently-preferred embodiment of the present invention.

### SUMMARY OF THE INVENTION

The present invention is directed to an apparatus that is useful for the effective implantation of viable cells or pharmaceutical formulations into the body of a patient. The apparatuses of the present invention preferably include a syringe with a reduced dead volume, a cannula adapted for cell implantation, and a rotational chamber. The syringe preferably has the capacity to contain a cellular suspension for administration to a patient. The apparatuses may include a motorized device which is adapted to rotate the syringe, cannula, and rotational chamber slowly. That rotation reduces cell clumping in the cellular suspension and better maintains the cellular suspension during the implantation procedure. In certain presently preferred embodiments, the syringe and cannula may be directly rotated without the addition of a rotational chamber. The present invention is particularly well suited for the implantation of neurons into the brain of a patient. In certain preferred embodiments, the apparatus may also be used to control the rate of cell delivery during implantation. The apparatuses of the present invention further provide for precise volumes of cell suspensions to be delivered to a patient. The apparatuses of the present invention preferably contain a minimal internal dead space to allow for accurate assessment of the volume of cells that are being administered to the patient. The apparatuses of the present invention also preferably fit into commercially available sterotactic frame systems currently used in human neurosurgery.

The present invention may also be used to delivery small volume fluid suspensions into tissues or cavities of the brain or other portion of the body. Through the cell or fluid delivery, the apparatuses of the present invention may be used to restore function of the target tissues in patients who have suffered ischemic, toxic, or traumatic injury to the target tissue.

### DETAILED DESCRIPTION OF THE INVENTION

It is to be understood that the figures and descriptions of the present invention have been simplified to illustrate elements that are relevant for a clear understanding of the invention, while eliminating, for purposes of clarity, other elements that may be well known. The detailed description will be provided hereinbelow with reference to the attached drawings.

The present invention encompasses apparatuses that are useful for the implantation of cells into target tissues within the body of a patient. While the present invention is described herein with particular reference to implanting neurons into the brain, numerous other applications are also considered to be within the scope of the present invention. For example, when employed to implant neurons into the brains of patients who suffer from Parkinson's disease, Huntington's disease, or who have had a stroke, the apparatuses of the present invention may be used to restore the function of the brain. When used with patients who have suffered a myocardial infarction, the apparatuses of the present invention may be used to restore function of the heart. Those of skill in the art will recognize further applications of the present invention to other tissues. The present invention may be used with human patients and animal patients and as either a therapeutic or experimental tool.

The present invention is preferably employed for the implantation of cells into the body of a patient. The present invention is particularly useful for the implantation of neurons into the brain of a patient. Specifically, the present invention may be used to implant cells into a stroke penumbra in a patient who has suffered a stroke.

Many previously-described cannulae may be used within the context of the present invention. For example, the cannula described in Kondziolka et al. (Cell Transplantation, 13:749-754, 2004; which is hereby incorporated by reference) may be employed simply within the context of a presently preferred embodiment described hereinbelow.

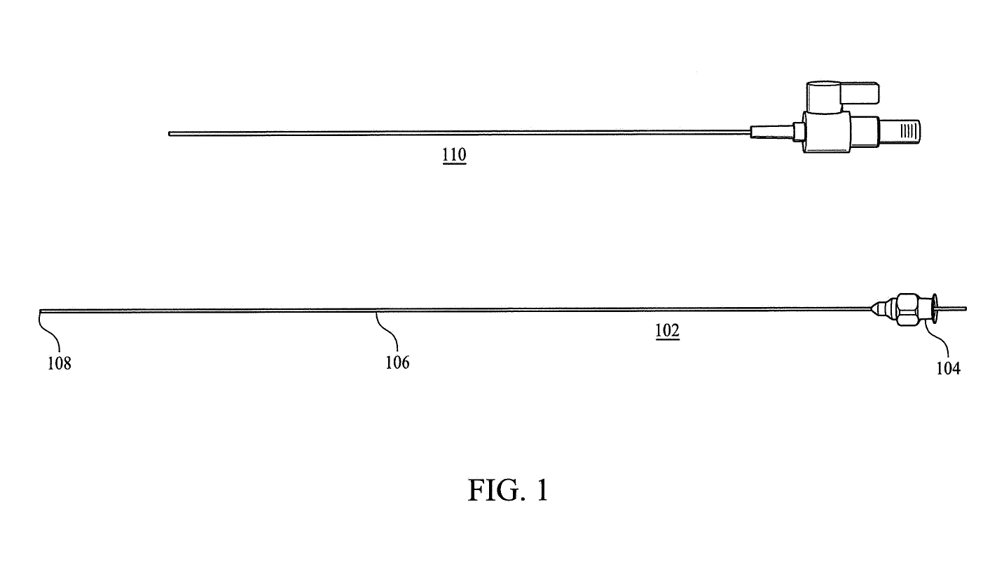

In certain presently preferred embodiments, the cell implantation cannula 102 is preferably fabricated of 100% stainless steel (type 304). Nickel-plated brass may be used for the hub 104. The outer diameter of the cannula 102 is preferably 890 microns and the internal bore diameter is preferably 250 microns. In certain embodiments, the internal volume of the cannula 102 is approximately 20 microliters to approximately 50 microliters. Due to its long shaft 106, the cannula 102 is typically flexible, thus potentially leading to difficulties when penetrating tissue. To reduce the difficulties that flexibility presents during penetration into target tissues, the cell implantation cannula 102 is preferably placed into a shorter, 15 cm rigid cannula 110 (called a stabilization cannula) which is first inserted into the patient to a point 4 cm proximal to the desired target. The cell implantation cannula 102 is placed through the stabilization cannula 110, and when placed to its full length, creates an instrument 19 cm in length, as shown in the preferred embodiment in FIG. 1. That size of cell implantation cannula 102 is particularly useful for implantation of cells into human brains when using Leksell stereotactic techniques.

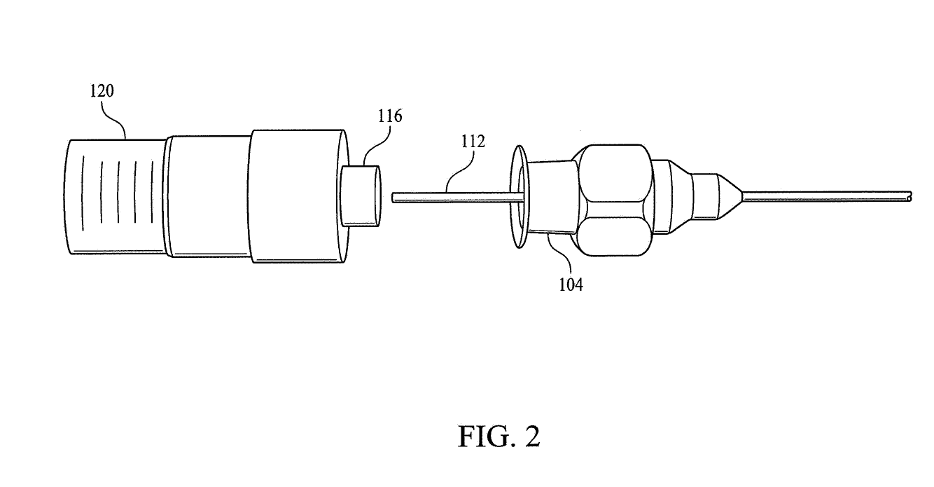

An additional feature of the present invention is the reduction or elimination of dead space within the hub 104. Cannulae for use in the present invention were designed to be used with a Hamilton-type syringe, though other designs may be employed. The cell implantation cannula 102 preferably has a rigid proximal extension 112 that connects directly with the luer connector 116 of the syringe 120 so that any fluid volume or cell suspension contained within the syringe 120 is passed directly into the cell implantation cannula 102, as shown in FIG. 2. The tip of the cell implantation cannula 108 is preferably gently rounded for reducing damage while the cell implantation cannula 102 is passing through the brain or other tissue. A narrow metal stylette is preferably removed from the cell implantation cannula 102 prior to passage of the fluid volume or cell suspension. The instrument may be sterilized using steam techniques or other conventional approaches.

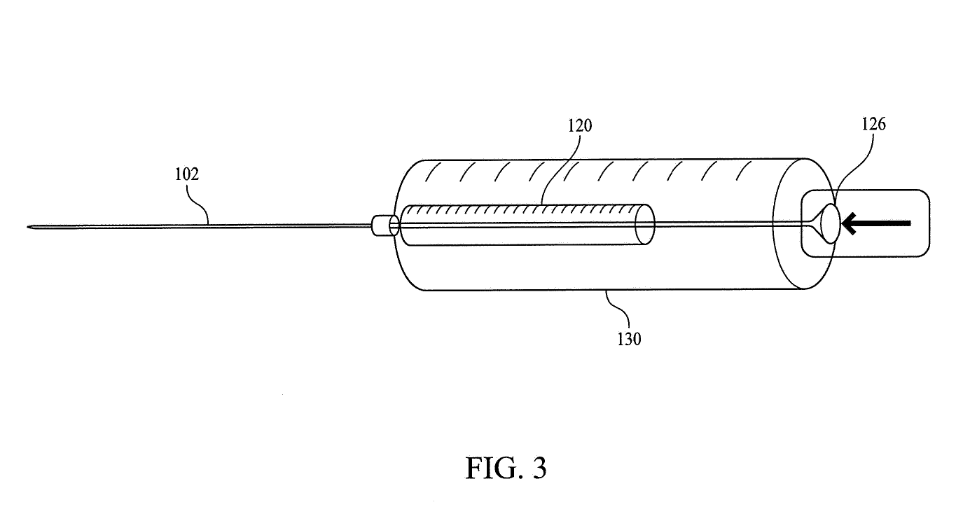

In presently preferred embodiments, the syringe 120 is placed inside of a rotational chamber 130 as shown in FIG. 3. In certain preferred embodiments, the rotation chamber 130 is transparent. The rotation chamber 130 is adapted so that it is able to be rotated around its central axis by a small motor or other mechanism commonly known to those of skill in the art. Preferably, the rotational movement of the rotation chamber 130 also revolves the syringe 120 and the cell implantation cannula 102. The rotation chamber 130 may be rotated at any desired rate, with a range of approximately 1 RPM to approximately 10 RPM being preferred and 6 RPM being most preferred. By rotating the assembly, the clumping of the cell suspension is reduced, thereby promoting the implantation of a more consistent suspension of vital cells.

The present invention also encompasses embodiments that do not include a rotation chamber 130. In those embodiments, the syringe 120 itself may be rotated by a motor either directly or indirectly.

In addition, presently-preferred embodiments of the present invention include a motorized plunger depressor for the controlled delivery of fluid or cell suspension from the cell implantation cannula 102 as shown in FIG. 3. In presently-preferred embodiments, the motorized plunger depressor is adapted to accept the plunger of the syringe 126 and to be able to depress the syringe plunger 126 at a controlled rate. The motorized plunge depressor may be driven by a linear actuator. In other presently preferred embodiments, other mechanisms may be used to depress the syringe plunger, such as a stepper motor or DC motor may be employed. The rate is preferably able to be controlled by the medical practitioner during the course of surgery.

The operation of the present invention will be described with reference to the following examples.

### EXAMPLE 1

One hour prior to implantation, the frozen LBS-neurons were thawed, gently washed twice in ISOLYTE S (McGaw Inc., Irvine, Calif.) and centrifuged at 200×g for 7 minutes at room temperature. The cell pellet was gently resuspended in ISOLYTE S. The viable cell count was determined with a sample of the LBS-neuron suspension using 0.4% Trypan blue and a hemocytometer. The cells were resuspended to a final concentration between 3.3×10⁷ and 4.0×10⁷ cells/mL in ISOLYTE S and aliquoted at volumes between 120 μL and 250 μL per sterile 1.0 mL vial. Depending upon the dose to be administered, one or more vials were prepared. Vials were loaded into a closed holder and carried by hand in an upright position to the operating room for immediate use.

Informed consent was obtained prior to the procedure for all patients. One week prior to surgery, all anticoagulant medications were discontinued. On the morning of surgery, the Leksell model G stereotactic coordinate frame was applied to the head under local anesthesia and mild sedation. A contrast-enhanced computed tomography (CT) scan was performed for stereotactic targeting. Five millimeter slices were obtained through the brain. Coronal and sagittal views were used to define a safe trajectory that entered a cortical gyrus and spared a sulcus. Stereotactic coordinates were obtained for each instrument placement—a point in the basal ganglia inferior to the stroke, within the mid-portion of the affected brain area, and in the superior aspect of the basal ganglia either within or beyond the stroke. For patients who received nine implants and 6 million cells, three trajectories were chosen in the same paramedian plane spaced by 5-6 mm at the target. During surgical planning, the LBS-neurons were thawed and processed. When the viable neuronal cells were formulated for dosing, they were brought to the operating room directly. Either a twist drill or burr hole craniostomy was fashioned under local anesthesia.

The dura mater was opened and a 1.8 mm outer diameter 15 cm long stabilizing cannula was inserted to a point 4 cm proximal to the final target. The stabilizing cannula had an internal volume of 100 microliters; the cell implantation cannula had a volume of 20 microliters. In the operating room, the cells were aspirated into a 250 microliter syringe. The internal volume of the cell implantation cannula was filled with the cell suspension. The cell implantation cannula was connected to a syringe inside of a transparent rotation chamber. The whole assembly was connected to a small motor that was adapted to rotate the rotation chamber, along with the syringe and cannulae. The plunger of the syringe was placed into a receptacle adapted to depress the plunger by another small motor. The cell implantation cannula was then inserted into the stabilizing cannula into the target tissue in the basal ganglia.

Once the cell implantation cannula, syringe, and rotation chamber assembly were connected to the small motor, rotation of the chamber was initiated at approximately 6 RPM. A 20 microliter volume of cells was injected over approximately 4 minutes at the first target site. The instrument was then withdrawn to the second and third sites for subsequent implants. After the three implantations were made, the cannulae were withdrawn from the brain and the wound either closed, or the next vial of cells opened to inject implants nos. 4-9 in those patients who received 6 million cells. Implants nos. 4-6 were delivered anterior to the first implants by 5-6 mm depending on stroke size, and implants nos. 7-9 were delivered 5-6 mm posterior to the first implants. Following surgery, a post-operative CT scan confirmed the absence of hemorrhage. All patients were discharged home the morning after surgery.

### EXAMPLE 2

The initial patient preparation was similar to Example 1. In Example 2, a point in the basal ganglia inferior to the center of the stroke was identified, and four other targets inferior to the stroke (anterior, posterior, medial, and lateral to the central target, usually spaced by 5 mm). For each of the five planned trajectories, the patient was to receive five cell implants spaced equally across a distance of 20-25 mm. Thus, each patient received their total cell dosage of 5 or 10 million cells divided into 25 implants. After the viable cells were formulated, they were brought to the operating room directly. A burr hole craniostomy was created under local anesthesia. The dura mater was opened and a 1.8 mm outer diameter 15 cm long stabilizing cannula was inserted to a point 4 cm proximal to the final target. A 0.9 mm outer diameter, 20 microliter internal volume cell implantation cannula was then inserted down to the deepest target point for the first implantation. In the operating room, the cells were aspirated into a 100 microliter syringe. The internal volume of the cell implantation cannula was filled with the cell suspension.

The cell implantation cannula was connected to a syringe inside of a transparent rotation chamber. The entire assembly was connected to a small motor that was adapted to rotate the rotation chamber, along with the syringe and cannulae. The plunger of the syringe was placed into a receptacle adapted to depress the plunger via another small motor. The cell implantation cannula was then inserted into the stabilizing cannula into the target tissue in the basal ganglia. After placement of the assembly into the chamber, rotation of the chamber was initiated at approximately 6 RPM. A 10 microliter volume of cells was injected over two minutes at the each target site by allowing a small motor to depress the plunger of the syringe at the specified rate.

The cannulae were then withdrawn to the more proximal targets along each trajectory. The total time for all implantations was approximately 150 minutes. After implantation, the wound was closed and a post-operative CT scan performed to confirm the absence of hemorrhage. All patients were discharged home the morning after surgery.

Those of skill in the art will recognize that numerous modifications of the above-described process can be performed without departing from the present invention, including the manner in which the syringe is rotated. For example, the tissue into which either cellular suspensions or pharmaceutical formulations are being injected may vary depending on the medical condition being treated. Further, one of skill in the art will recognize multiple manners in which depression of the plunger or rotation of the assembly may be implemented.

I claim:

1. An apparatus for the implantation of cells into a patient, comprising:
a cell implantation cannula;
a syringe comprising a plunger, operably connected to said cell implantation cannula; and
a first motorized device, wherein said motorized device is adapted to rotate said syringe and said cell implantation cannula.

2. The apparatus of claim 1, further comprising a second motorized device adapted to depress said plunger.

3. The apparatus of claim 2, wherein said second motorized device includes a linear actuator adapted to depress said plunger.

4. The apparatus of claim 1, further comprising a stabilization cannula, wherein said cell implantation cannula is adapted to fit inside said stabilization cannula.

5. The apparatus of claim 1, wherein said cell implantation cannula has an internal volume of approximately 20 microliters to approximately 50 microliters.

6. The apparatus of claim 1, wherein said syringe has a small dead volume.

7. The apparatus of claim 1, further comprising a rotational chamber, wherein said rotational chamber is adapted to accept said syringe.

8. A method of administering a cellular suspension into a target tissue of a patient, comprising the steps of:
connecting a cell implantation cannula to a syringe that includes a plunger, wherein said syringe contains a cellular suspension;
inserting said cell implantation cannula into said target tissue;
rotating said syringe and said cell implantation cannula; and
depositing said cellular suspension into said target tissue.

9. The method of claim 8, wherein said depositing step includes depressing said plunger.

10. The method of claim 9, wherein said depressing said plunger is accomplished by a mechanical device.

11. The method of claim 10, wherein said mechanical device includes a linear actuator.

12. The method of claim 8, wherein said rotating is accomplished using a mechanical device.

13. The method of claim 12, wherein said motorized device accomplishes said to rotate said syringe, said cell implantation cannula, and said rotational chamber at a rate of between 1 RPM and 10 RPM.

14. The method of claim 13, wherein said rate is approximately 6 RPM.

15. The method of claim 8, further comprising the step of placing said syringe in a rotational chamber.

16. The method of claim 15, wherein said rotating step further comprises rotating said rotational chamber.

17. The method of claim 8, wherein said delivering occurs at approximately 5 microliters per minute.

18. The method of claim 8, wherein said target tissue is brain.

19. The method of claim 8, further comprising the step of inserting a stabilization cannula into said target tissue before said inserting said cell implantation cannula into said target tissue.

20. The method of claim 19, wherein said cell implantation cannula is inserted into said target tissue through said stabilization cannula.

21. The method of claim 19, wherein said cell implantation cannula is longer than said stabilization cannula.
